(19) United States
(12) Patent Application Publication
(10) Pub. No.: US 2010/0128840 A1
(43) Pub. Date: May 27, 2010

(54) PHOTOGRAPHING APPARATUS OF COMPOSITION-IMAGE FOR DENTAL DIAGNOSIS

(76) Inventor: Young-Jin Cha, Seoul (KR)

Correspondence Address: Law Office of ROBERT C. KLINGER, 2591 Dallas Parkway, Suite 300, FRISCO, TX, 75034, US

(21) Appl. No.: 12/451,809

(22) PCT Filed: May 27, 2008
(86) PCT No.: PCT/KR2008/002960

§ 371 Date: Nov. 30, 2009

(30) Foreign Application Priority Data
May 29, 2007 (KR) 10-2007-0051893

(51) Int. Cl. A61B 6/14; H05G 1/60

(52) U.S. Cl. 378/4; 378/38

(57) ABSTRACT

A composite imaging apparatus for dental diagnosis, wherein dental diagnosis for teeth/periodontal diseases and orthodontics can be simply done even with an imaging apparatus, and a patient's head portion can be automatically rotated according to image taking directions so as to simplify radiography, save radiography time, and minimize X-ray exposure. The apparatus includes a rotary arm horizontally rotating in left and right directions in order to take an X-ray image of teeth, jawbone and alveolar bone of a patient; a support frame vertically moving with a proper range according to a height of the patient, supporting and enabling the rotary arm to be fixed and horizontally rotate; and an object moving device formed on the base so as to move up and down a for certain range, reciprocate in forward and backward directions, horizontally rotate in forward and reverse directions while the patient is sitting thereon, and facilitate radiography.

### TECHNICAL FIELD

The present invention relates to a composite imaging apparatus for dental diagnosis, and more particularly, to a composite imaging apparatus for dental diagnosis, wherein a dental diagnosis for teeth/periodontal diseases and orthodontics can be simply done even with an imaging apparatus, and a patient's head portion can be automatically rotated according to image taking directions so as to simplify radiography, save radiography time, and minimize X-ray exposure.

### BACKGROUND ART

In the dentistry, an imaging apparatus is generally provided to diagnose the conditions of the alveolar bone including the teeth for the cure of the teeth/periodontal disease and the orthodontics.

The main objective of the X-ray imaging apparatus is for the dentist to consider any existing diseased factors in the patient's jawbone and information affecting the medical treatment together with the recognition of the size/shape of the jawbone and the anatomical changes of the skull along with the vertical and horizontal axis, and those image information are very important to the most suitable dental treatment and every procedures for increasing the long-term success after the treatment.

Related imaging methods generally emit an X-ray beam, and available examples thereof include a panoramic X-ray image system, which is a two-dimensional flat image of the jawbone including the teeth; a computerized tomography image (CT) system, which is a comparatively more accurate three-dimensional image; and a cephalometric imaging system, in which the images of the patient's head can be taken from front to back, from back to front, from the left, or right side.

FIG. 1 is an overall perspective view illustrating the structure of a conventional dental X-ray imaging apparatus of the prior art. The conventional dental X-ray imaging apparatus 900 is provided with a panoramic imaging system 901 and a cephalometric image detector portion 950. The panoramic imaging system 901 includes a base 910 standing on the floor of the building, a main body including a support column 911 supported by the base 910 and an elevating portion 920 mounted to the support column 911 so as to be movable upward and downward along the support column 911, provided with a top frame 930 installed horizontally on an upper end of the elevating portion 920, a rotary arm 940 installed rotatably on one end of the top frame 930, an X-ray generator 942 formed on one side of the rotary arm 940, and a panoramic image detector 944 formed on the other side of the rotary arm 920, for detecting a generated X-ray by. The cephalometric image detector portion 950 includes a support arm 951 installed on the elevating portion 920, a support frame 953 supported by the support arm 951, and a cephalometric detector 954.

Further, since the panoramic image detector 944 is removably coupled to the rotary arm 940, in the case where cephalometric images are intended to be taken, the panoramic image detector 944 is separated from the imaging apparatus, so that X-ray beams emitted from the X-ray generator 942 can be detected on the cephalometric image detector 954.

Since the cephalometric image detector 954 installed on the panoramic image apparatus 901 requires that the X-ray beams for taking the image of the head be emitted at a little further location than for the panoramic image, a support arm 951 having a proper length can be formed with a regular interval from the X-ray generator 942. For that reason, the indoor space occupied by the imaging system 900 needs to be expanded despite the imaging system 900 is not being used and the reliability of the image quality can be lowered because of the rough shaking when mechanical and/or electrical vibration in the cephalometric image-taking process is transferred to the cephalometric image detector 954 via the support arm 951.

Since radiographies including the teeth, the alveolar bone and the skull's jawbone of an object (patient) are being processed only by the simple rotation of the rotary arm 940 in the leftward and rightward direction while the patient is positioned on the base 910, there can be some problems such as an inefficient image-taking process, for example, in which the patient has to change his/her pose according to the body portion, the image of which is being taken, and resultant time delay.

In addition, the cephalometric image detector 950 is not rotatable as mentioned above and is installed apart from the X-ray generator 942 with a certain interval, and there has been a cumbersome job of removing the panoramic image detector 944 for the cephalometric image taking process.

### DISCLOSURE OF INVENTION

### Technical Problem

The present invention has been made to solve the above mentioned problems occurring in the prior art, and therefore, the present invention is directed to a composite imaging apparatus for dental diagnosis, wherein respective detectors for a CT image, a panoramic image and a cephalometric image are coupled to proper positions of a single rotary arm, so that radiographies for various kinds of dental diagnosis can be simply and efficiently implemented.

The present invention is also directed to a composite imaging apparatus for dental diagnosis, wherein an object-moving device, which can rotate by 360 degrees and also move in downward and upward directions and frontward and backward directions, is additionally provided on the base, where an object (patient) is located for the radiography, to rotate and move in opposite directions with respect to the rotary arm, so as to reduce X-ray imaging time and thus minimize X-ray exposure, thereby maximizing the efficiency of a dental treatment.

### Technical Solution

According to an aspect of the present invention, there is provided a composite imaging apparatus for dental diagnosis. The composite imaging apparatus includes a rotary arm horizontally rotating in left and right directions in order to take an X-ray image of teeth, jawbone and alveolar bone of a patient; a support frame vertically moving with a proper range according to a height of the patient, supporting and enabling the rotary arm to be fixed and horizontally rotate; and an object moving device formed on the base so as to move up and down a for certain range, reciprocate in forward and backward directions, horizontally rotate in forward and reverse directions while the patient is sitting thereon, and through systematic operation with the rotary arm, facilitate radiography.

### Advantageous Effects

As clearly understood in the above explanation, the composite imaging apparatus for dental diagnosis in accordance with the present invention has the cephalometric image detector, which is provided as a unitary part on the rotary arm, laterally spaced apart from and positioned lower than the CT and panoramic image detector on the rotary arm. This structure can resolve the cumbersome of equipment usage of the prior art, in which the cephalometric image detector was installed due to its nature on the elevating frame, separate from the rotary arm, and thereby maximize cost saving and the efficiency of equipment usage for radiography.

Further, the position of the patient's head can be automatically changed by moving the object moving device, so that various kinds of radiography can be easily done without taking some annoying actions, such as the patient needs to change his/her pose according to an instruction from a nurse or a surgeon while standing on the base of the conventional X-ray imaging apparatus. The object moving device can be elevated up and down, moved forward and backward, or rotated for 360 degrees in a direction opposite the movement of the rotary arm, so that the radiography time can be dramatically reduced and the X-ray exposure harmful to the human body can be minimized at the same time.

### BRIEF DESCRIPTION OF THE DRAWINGS

FIG. 1 is an overall perspective view illustrating the structure of a conventional dental X-ray imaging apparatus of the prior art.

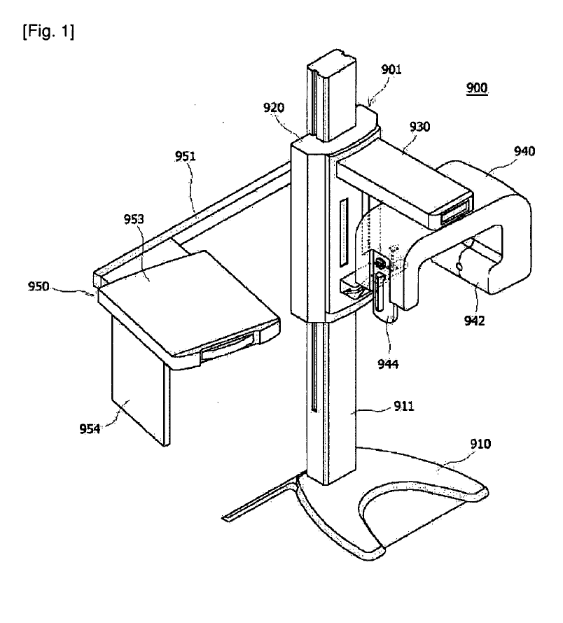

FIG. 2 is an overall perspective view showing the structure of a composite imaging apparatus for dental diagnosis in accordance with the present invention.

FIG. 3 and FIG. 4 are cross-sectional views showing the structure of the base portion of the composite imaging apparatus for dental diagnosis in accordance with the present invention, taken from the side thereof.

FIG. 5 is a front elevational view showing the structure of a rotary arm as an important part of the composite imaging apparatus for dental diagnosis in accordance with the present invention.

FIG. 6 is an exploded diagram illustrating the structure of an object moving device in the composite imaging apparatus for dental diagnosis in accordance with the present invention.

FIG. 7 and FIG. 8 are exemplary diagrams illustrating the operations of the composite imaging apparatus for dental diagnosis in accordance with the present invention.

### MAJOR REFERENCE NUMERALS AND SYMBOLS OF THE DRAWINGS

A: imaging apparatus 1: rotary arm

1a: head positioning space 2: support frame

3: object moving device 11: X-ray source

12: CT and panoramic image detector

13: cephalometric image detector

21: elevating frame 22: support column

23: base housing 23a: fitting space

31: moving means 32: treatment chair

311: turntable 312: support shaft

312a: ball bearing 313: bevel gear

314: driving motor 315: reciprocating bed

315-1: moving plate 315-2: fixed plate

316: horizontal shock absorber

317: vertical shock absorber

321: chin rest

### BEST MODE FOR CARRYING OUT THE INVENTION

Exemplary embodiments of the present invention will be described below in detail with reference to the accompanying drawings so as to be easily implemented by those skilled in the art.

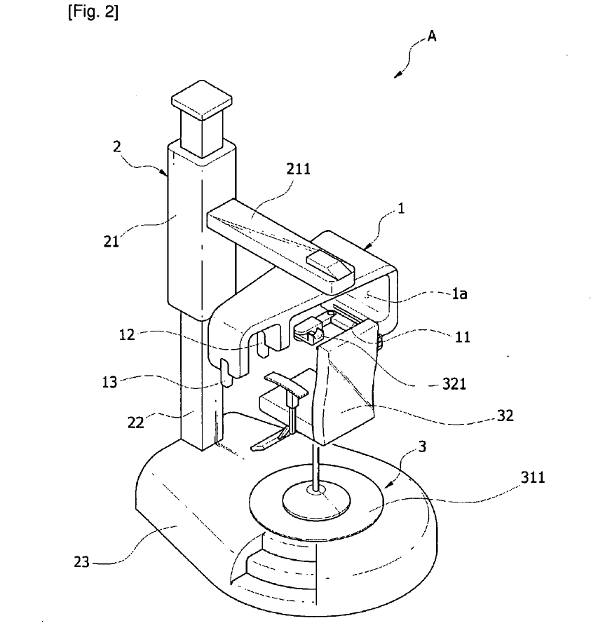

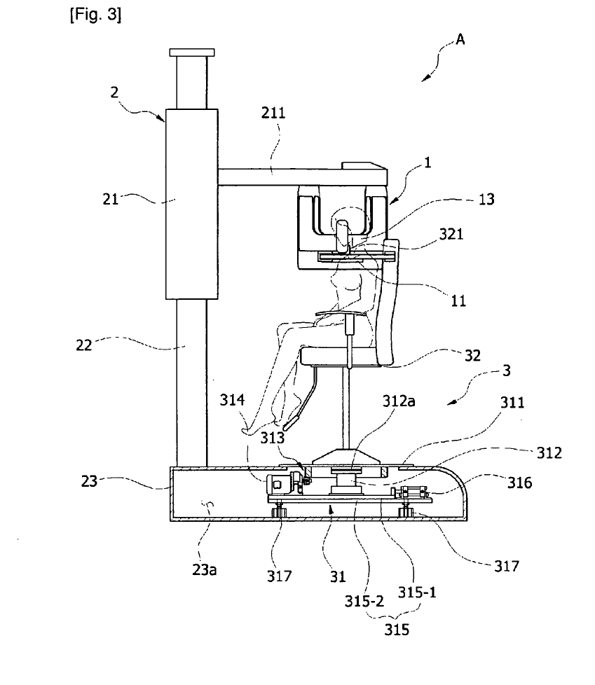

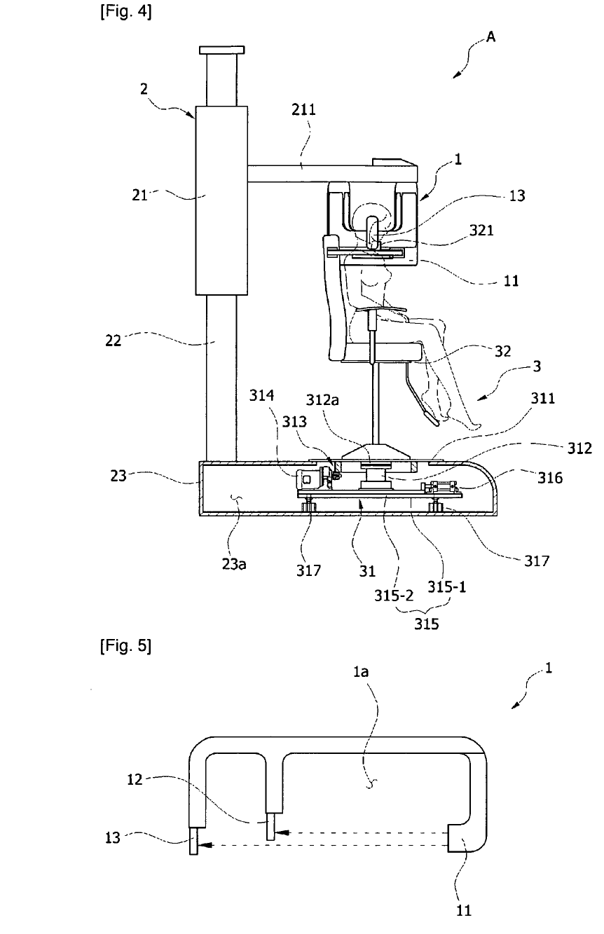

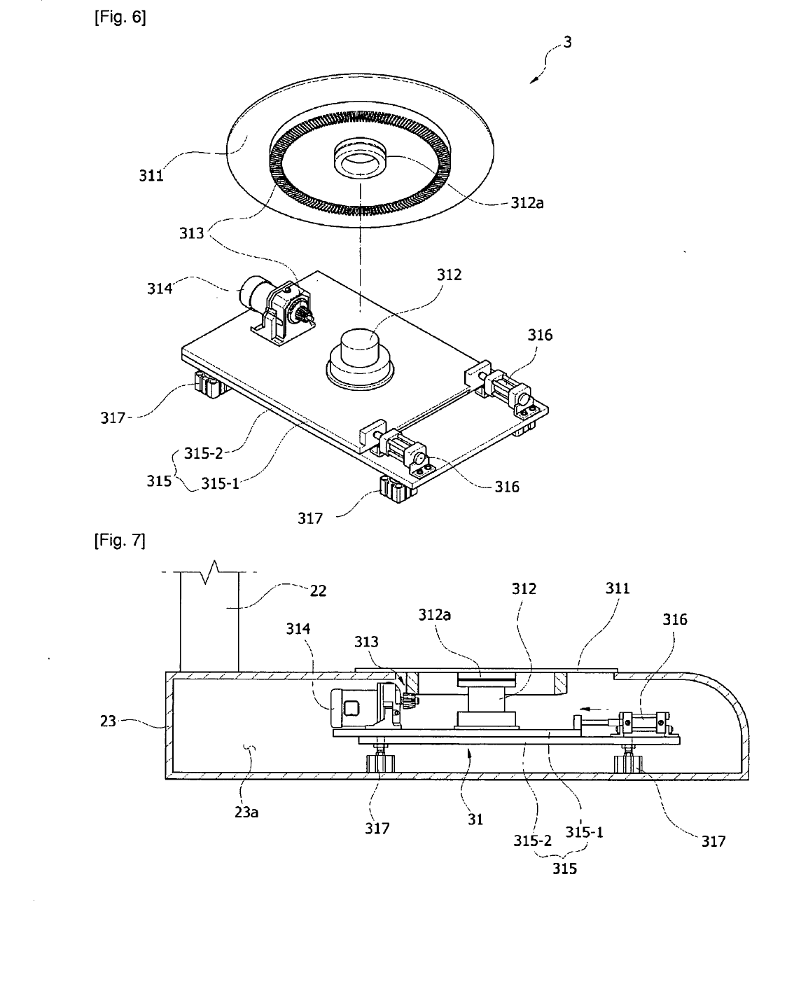

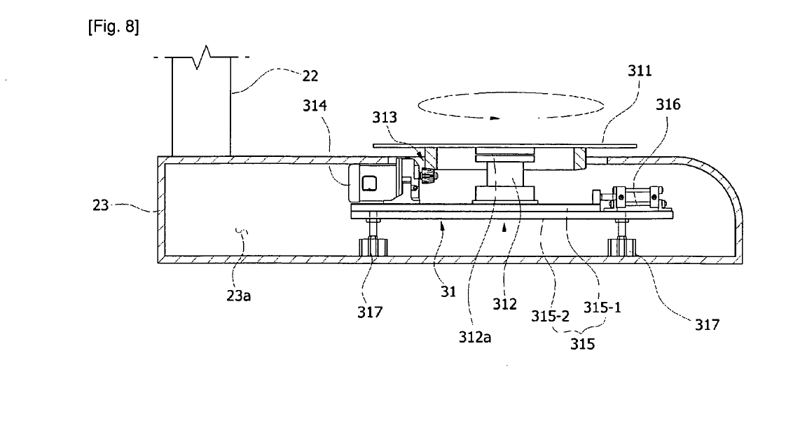

FIG. 2 is an overall perspective view showing the structure of a composite imaging apparatus for dental diagnosis in accordance with the present invention, FIG. 3 and FIG. 4 are cross-sectional views showing the structure of the base portion of the composite imaging apparatus for dental diagnosis in accordance with the present invention, taken from the side thereof, FIG. 5 is a front elevational view showing the structure of a rotary arm as an important part of the composite imaging apparatus for dental diagnosis in accordance with the present invention, FIG. 6 is an exploded diagram illustrating the structure of an object moving device in the composite imaging apparatus for dental diagnosis in accordance with the present invention, and FIG. 7 and FIG. 8 are exemplary diagrams illustrating the operations of the composite imaging apparatus for dental diagnosis in accordance with the present invention.

Referring to FIGS. 2 to 4, the composite imaging apparatus for dental diagnosis A in accordance with the present invention includes a rotary arm 1, a support frame 2 and an object moving device 3. The rotary arm 1 horizontally rotates in left and right directions for the purpose of dental diagnosis such as radiography. The support frame 2 moves vertically with the proper range according to the patient's height while supporting and enabling the rotary arm 1 to be fixed and horizontally rotate. The object moving device 3 is formed on the base; and can move up and down a for certain range, reciprocate in forward and backward directions, horizontally rotate in forward and reverse directions while the patient is sitting thereon; and through systematic operation with the rotary arm 1, facilitate the radiography.

The rotary arm 1 is to take the X-ray images of the alveolar bone and the jawbone of the skull, including the teeth, from various directions. As shown in FIG. 5, the rotary arm 1 is coupled at the upper center thereof with the support frame 2 by the hinge so as to rotate 360 degrees in left and right directions, and is provided with an overturned U-shaped (∩-shaped) structure with a head positioning space 1a covering the patient's head. An X-ray source 11 is provided in one end of the rotary arm 1, with a collimator for emitting X-ray beams, and a CT and panoramic detector 12 is formed in the other end of the rotary arm 1 so as to be used for the two or three dimensional radiography of the teeth and/or the alveolar bone for the purpose of the dental diagnosis of the teeth and/or the alveolar bone. For the radiography of the skull's jawbone which needs the dental diagnosis for the orthodontics, a cephalometric image detector 13 is additionally installed in an outer end, which is further elongated than the other end of the rotary arm 1.

Since the cephalometric image detector 13 is needed for the extended emitting range due to its nature, the cephalometric image detector 13 is installed more or less farther than the CT and panoramic image detector 12 and, at the same time, is preferable to be located comparatively lower than the CT and panoramic image detector 12 so that the X-ray beam emitted from the X-ray source 11 is not disturbed by the CT and panoramic image detector 12.

A support frame 2 is coupled with the rotary arm 1 by the hinge allowing the rotary arm 1 to move and rotate for 360 degrees, and is designed to move vertically with the proper range according to the patient's height. The support frame 2 is constructed with an elevating frame 21 capable of vertically moving up and down in a proper range, with a fixed frame 211 formed on one side thereof for the fixing and rotation of the rotary arm 1; a support column 22 supporting and enabling the fixed frame 21 to vertically move; and a base housing 23 coupled with the bottom of the support column 22 as one body, the base housing 23 having an inner holding space 23a for the object moving device 3 to be housed therein.

The object moving device 3 is to save the radiography time by moving reversely to the rotation of the rotary arm 1, which acts to take CT images, panoramic images and cephalometric images. As shown in FIG. 3, FIG. 4, and FIG. 6, the object moving device 3 includes a moving means 31, which is installed in the holding space 23a of the base housing 23, for automatically causing the patient's body to rotate in forward, backward, leftward and rightward rotations or move in upward and downward directions; and a treatment chair 32 designed for the patient to sit conveniently so as to prevent the patient's body from shaking due to the movement of the moving means 31.

The moving means 31 is designed to rotate the patient's body reversely from the rotary arm 1, which acts to take the X-ray images of the patient's head by rotating around the patient's head. The moving means 31 includes a turntable 311, a bevel gear 313, a driving motor 314, a reciprocating bed 315, a horizontal shock absorber 316 and a plurality of vertical shock absorbers 317. The turntable 311 can feely change the direction of the patient's body sitting on the treatment chair 32. The support shaft 312 is vertically coupled with the turntable 311 at the central bottom to rigidly fix the turntable 311 and guide the movement of the turntable 311. The bevel gear 313 controls the leftward/rightward rotation of the turntable 311 for 360 degrees. The driving motor 314 enables the bevel gear 313 to rotate by gear engagement. The reciprocating bed 315 is composed of a moving plate 315-1 and a fixed plate 315-2 coupled to the moving plate 315-1. The moving plate 315-1 slides to guide the forward/backward movement of the turntable 311, with the support shaft 312 and the driving motor 314 coupled to the upper surface thereof. The horizontal shock absorber 316 is coupled to the upper portion of the fixed plate 315-2 in order to horizontally move the moving plate 315-1 in the frontward and backward directions by a cylinder rod. The vertical shock absorbers 317 are coupled to underside edges of the fixed plate 315-2, allowing the turntable 311 to move in upward and downward directions by vertically moving the reciprocating bed 315 through the upward/downward movement of the cylinder rod.

Meanwhile, on the upper part of the support part 312, a ball bearing 312a is coupled to help the turntable 311 smoothly move for horizontal 360 degrees through the bevel gear 313.

Further, a chin rest 32 is provided with a separate frame, extending from the back of the treatment chair 32 to the patient's chin area, for the patient to place his/her chin so as not to move the head.

Below, a detailed description will be given of the operations of the composite imaging apparatus for dental diagnosis formed with the above mentioned construction in accordance with the present invention.

First, referring to FIGS. 2 to 4, a patient is seated on the treatment chair 32 of the object moving device 3 of the X-ray imaging apparatus of the invention, which is designed for the dental diagnosis of the alveolar bone and jawbone including teeth in order to treat teeth/periodontal diseases and orthodontics using X-ray.

Next, a dental diagnosis is performed by placing the chin of the patient, seated in the treatment chair 32, to the chin rest 321, which is formed in consideration of the patient's body structure, and by stopping the movement of the patient body according to an instruction from a nurse or a surgeon in the dental clinic.

After that, a panoramic imaging is generally taken to diagnose the conditions of the teeth and the alveolar bone of the patient who needs a nerve therapy due to a tooth disease such as a decayed tooth or a periodontal disease such as inflamed gums, and a CT imaging, which can take more detailed three-dimensional images than the panoramic imaging, is used when the implant surgery is needed.

For the CT and panoramic imaging, as shown in FIG. 6, the X-ray images will be taken for the related area from the various directions when the X-ray beam (electromagnetic wave) from the X-ray source 11 reaches the CT and panoramic image detector 12 after having penetrated the head, as soon as the rotary arm 1 rotates 360 degrees in the left/right direction at the outside of the patient's head area

At this time, as shown in FIG. 7, the CT and panoramic image taking can be implemented fast and efficiently by rotating the turntable 11 in the reverse direction against the turning direction of the rotary arm 1 while the bevel gear 313 is moving by engaging with the driving motor 314 of the moving means 31.

On the other hand, when the orthodontics is needed to cure for the imbalanced jaw structure like a jutting chin (referred to as mandibular prognathism), a cephalometric image taking process, in which the whole skull can be examined, is implemented to diagnose the situation of the patient's jawbone.

For the cephalometric image taking process as described above, an X-ray image can be taken for the related area when the X-ray beam from the X-ray source 11 reaches the cephalometric image detector 13 after having penetrated the head in the sate where the rotary arm 1 around the patient's head has stopped moving.

In the mean time, as shown in FIG. 5, since the whole structure of the patient's skull and jawbone needs to be taken for the cephalometric image taking process, the emitting distance from the X-ray source 11 in the cephalometric image taking process should be longer than in the CT and panoramic image taking as mentioned above. The cephalometric image detector 13 is located more or less farther and comparatively lower than the CT and panoramic image detector 12 so that images can be efficiently taken without being disturbed.

Then, as shown in FIG. 8, the cephalometric image taking process can be efficiently implemented to take the X-ray images for the whole structures of the head and jawbone by moving the moving plate 315-1 of the reciprocating bed 315 in the forward and backward directions with the actuation of the horizontal shock absorber 316 or by moving the turntable 311 in the forward and backward directions or the upward and downward directions with the actuation of the vertical shock absorber 317, while the patient can stay sitting on the treatment chair 32 without any cumbersome actions such as moving his/her head or changing the direction by moving his/her body.

While the present invention has been described with reference to the particular illustrative embodiments and the accompanying drawings, it is not to be limited thereto. Accordingly, the foregoing embodiments can be suitably modified and altered, and such applications fall within the scope and spirit of the present invention that shall be defined by the appended claims.

1-8. (canceled)

9. A composite imaging apparatus for dental diagnosis, comprising:
a rotary arm horizontally rotating in left and right directions in order to take an X-ray image of teeth, jawbone and alveolar bone of a patient;
a support frame vertically moving with a proper range according to a height of the patient, supporting and enabling the rotary arm to be fixed and horizontally rotate;
an object moving device formed on the base so as to move up and down a for certain range, reciprocate in forward and backward directions, horizontally rotate in forward and reverse directions while the patient is sitting thereon, and through systematic operation with the rotary arm, facilitate radiography;
wherein the object moving device includes a moving means for automatically causing a body of the patient to rotate in forward, backward, leftward and rightward or move in upward and downward directions, and a treatment chair designed for the patient to sit conveniently so as to prevent the body of the patient from shaking due to movement of the moving means; and
wherein the moving means includes a turntable for feely changing a body direction of the patient sitting on the treatment chair, a support shaft vertically coupled with the turntable at a central bottom to rigidly fix the turntable and guide a movement of the turntable, a bevel gear controlling leftward and rightward rotation of the turntable for 360 degrees, a driving motor enabling the bevel gear to rotate by gear engagement, a reciprocating bed including a moving plate and a fixed plate coupled to the moving plate, wherein the moving plate slides to guide forward and backward movement of the turntable, with the support shaft and the driving motor coupled to an upper surface thereof, a horizontal shock absorber coupled to an upper portion of the fixed plate in order to horizontally move the moving plate in frontward and backward directions by a cylinder rod, and a plurality of vertical shock absorbers coupled to underside edges of the fixed plate, allowing the turntable to move in upward and downward directions by vertically moving the reciprocating bed through upward and downward movement of the cylinder rod.

10. The composite imaging apparatus for dental diagnosis according to the claim 9, wherein the rotary arm is coupled at the upper center thereof with the support frame by the hinge so as to rotate 360 degrees in left and right directions, and is provided with an overturned U-shaped structure with a head positioning space covering a head portion of the patient;
an X-ray source is provided in one end of the rotary arm, and has a collimator for emitting X-ray beams;
a CT and panoramic detector is formed in another end of the rotary arm so as to be used for two or three dimensional radiography of the teeth and/or the alveolar bone for dental diagnosis of the teeth and/or the alveolar bone; and
a cephalometric image detector is additionally installed in an outer end, which is further elongated than the other end of the rotary arm, for radiography of the jawbone of a skull which needs a dental diagnosis for orthodontics.

11. The composite imaging apparatus for dental diagnosis according to the claim 9, wherein the support frame includes an elevating frame capable of vertically moving up and down in a proper range, with a fixed frame formed on one side thereof for the fixing and rotation of the rotary arm, a support column supporting and enabling the fixed frame to vertically move, and a base housing coupled with the bottom of the support column as one body and having an inner holding space for the object moving device to be housed therein.

12. The composite imaging apparatus for dental diagnosis according to the claim 10, wherein the cephalometric image detector is installed more or less farther and comparatively lower than the CT and panoramic image detector and so that the X-ray beam emitted from the X-ray source is not disturbed by the CT and panoramic image detector.

13. The composite imaging apparatus for dental diagnosis according to the claim 9, further comprising a chin rest having a frame extended from the back of the treatment chair to a chin area of a patient, for the patient to place the chin so as not to move a head portion.

14. The composite imaging apparatus for dental diagnosis according to the claim 9, further comprising a ball bearing coupled to an upper part of the support shaft to help the turntable smoothly move for horizontal 360 degrees through the bevel gear.
